(19) United States
(12) Patent Application Publication
(10) Pub. No.: US 2007/0005021 A1
(43) Pub. Date: Jan. 4, 2007

(54) Magnetic plunger rod re-setting mechanism and plunger rod lock

(76) Inventors: Philippe Kohlbrenner, Kaltacker (CH); Benjamin Reinmann, Madiswil (CH); Roney Graf, Burgdorf (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/478,310

(22) Filed: Jun. 29, 2006

(30) Foreign Application Priority Data
Jul. 1, 2005 (DE) 20 2005 010 389.6

(51) Int. Cl. A61M 5/00; B65D 88/54; G01F 11/00

(52) U.S. Cl. 604/208; 604/135; 222/333; 222/340

(57) ABSTRACT

An injection device which can accommodate an ampoule, the device including a plunger rod which can be moved out of an initial position in a dispensing direction and is thus able to act on a plunger associated with the ampoule, a magnetic member whereby the plunger rod can be moved along its longitudinal axis relative to the magnetic member, a magnetic counter-member disposed on the plunger rod, wherein the magnetic member and the magnet counter-member magnetically co-operate in at least one position of the plunger rod.

### CROSS-REFERENCE TO RELATED APPLICATION

This application claims priority to German Application No. DE 20 2005 010 389.6, filed Jul. 1, 2005, the content of which is incorporated in its entirety herein by reference.

### BACKGROUND

The present invention relates to devices for delivering, dispensing, injecting or administering substances, and to methods of making and using such devices. More particularly, it relates to injection devices, and to an injection device, more particularly an injection pen, with a magnetic plunger rod re-setting mechanism and a magnetic plunger rod lock.

In many injection devices, to change an ampoule or to insert such an ampoule, a driving means acting on a piston has to be moved back into an initial position. Depending on the drive and dose-metering mechanism used, there may be a risk that the drive means for a plunger is not fully retracted into the housing of the device. This can lead to the risk of an overdose, which can have serious complications for a patient.

A needleless injection gun is known from the prior art and is described in patent specification WO 03/068296 A3. A container containing the product to be administered is fitted onto the gun and serves as a syringe, from which the product to be administered can be delivered to a chamber via a flexible pipe. A plunger acts on the product contained in the chamber and is driven by a bolt in order to dispense product. To dispense product, the bolt is moved in the direction of the plunger by the impact of compressed air. Once the product has been dispensed, the bolt is returned to its initial position and is retained in the initial position by means of a magnet.

Patent specification GB 2 342 047 A discloses a syringe with a base body and a plunger longitudinally guided in the base body. An injection needle provided for administering an injection and projecting in the distal direction of the syringe is attached to a base, which is mounted so that it can be moved lengthways by the syringe base body. The base and the plunger rod each have magnets, which impact with one another after an injection so that they magnetically stick to one another. Pulling back the plunger causes the base with the needle to be pulled back into the syringe base body so that there is no longer any risk of injury by the injection needle.

### SUMMARY

An object of the present invention is to provide an injection device, such as an injection pen, which reduces or prevents the chance of an overdose of a product to be administered.

In one embodiment, the present invention comprises an injection device comprising a plunger rod moveable from an initial position in a dispensing direction and having a longitudinal axis, a magnetic member whereby the plunger rod can be moved along the longitudinal axis relative to the magnetic member, a magnetic counter-member associated with the plunger rod, wherein the magnetic member and the magnet counter-member magnetically co-operate in at least one position of the plunger rod.

In one embodiment, the present invention comprises an injection device, e.g., an injection pen, in which an ampoule can be inserted. The ampoule contains a product intended for administration, e.g., a medicinal fluid or substance, and is closed off at one end by a plunger which can be moved longitudinally in the ampoule. The injection device has a plunger rod, which can be moved from an initial position in a dispensing direction. The dispensing direction is the direction in which the plunger has to be moved in the ampoule to force the product out of the ampoule. The dispensing direction is generally along or parallel to the longitudinal axis of the injection device. The initial position of the plunger rod may be a position in which the ampoule is removed and replaced by another ampoule, for example, or in which the plunger rod assume its most proximal (i.e., rear) position, namely in a proximal end abutment. The initial position of the plunger rod may also be such that when an ampoule is inserted, the plunger rod does not act on the plunger. By “proximal” is meant the direction pointing towards a user and by “distal” (i.e., front) is meant the direction pointing away from the user or the direction in which the needle provided for an injection points.

The injection device also has a magnetic means and a magnetic counter-means. In some preferred embodiments, at least one of the magnetic means and magnetic counter-means comprises a magnet. A magnet might be an electromagnet, for example, or, in some preferred embodiments, a permanent magnet. By permanent magnet is meant a magnet which forms a magnetic field without the influence of an externally induced magnetic field. The other of the magnetic means and counter-magnetic means may be made from a ferromagnetic material, for example, such as iron, cobalt, nickel or an alloy containing at least one of iron, nickel, cobalt, for example. By ferro-magnet or ferromagnetic material is meant a material which is itself magnetised under the influence of an externally induced magnetic field and which is no longer magnetic or only weakly magnetic when the externally induced magnetic field is removed. In another embodiment, both the magnetic means and magnetic counter-means may be a magnet. If two magnets are moved towards one another by means of the same poles, such as North pole-North pole or South pole-South pole, for example, they will repel each other and if moved towards one another by opposite poles, such as North pole-South pole for example, they will be attracted to one another.

In one embodiment of the present invention, the plunger rod is displaceable along its longitudinal axis relative to the magnetic means. The magnetic means may be fixedly attached to the housing for example, or at least disposed so that it does not move with the plunger rod as it moves. For example, the magnetic means may also be disposed on a part which is displaceable relative to the housing to a limited degree.

In some preferred embodiments, the magnetic counter-means is disposed on the plunger rod. For example, the magnetic counter-means may be screwed to, adhered to the plunger rod, enclosed by the plunger rod or pressed into an enclosure, or connected by other suitable, structures or methods. Since it may be of advantage to ensure that a permanent magnet is exposed to as little vibration as possible, at least one damping means may be provided, which provides a springing or compensating action (i.e., damps vibrations) for at least one of the magnetic means and magnetic counter-means in the event of an abrupt impact of the two. The magnetic means may also be disposed on a threaded nut cap, or pushed into it. A spring may also be used for this purpose. In a preferred embodiment, the magnetic means is spring-mounted, e.g., on a dose-metering element. A guide sleeve connected to the plunger rod may also enclose the magnetic counter-means, at least around its periphery, or also enclose it in the axial directions, in order to protect the magnetic counter-means from damage or vibrations. The magnetic counter-means sits in the guide sleeve in a push-fit arrangement. Alternatively or in addition, the magnetic means and/or the magnetic counter-means may be provided with a protective coating, e.g., one which has a damping action. It may be of advantage to use an elastomeric coating for this purpose. In a preferred embodiment, the plunger rod is the magnetic counter-means. This being the case, in some preferred embodiments, the magnetic counter-means is ferromagnetic and the magnetic means is a magnet.

In one embodiment in which the magnetic means and the magnetic counter-means are both magnets, it may be of advantage to dispose them so that the magnetic means and magnetic counter-means attract or repel. In its initial position relative to the position which it assumes after dispensing the product, the plunger rod is pushed proximally. As mentioned above, the initial position may be the most proximal position of the plunger rod. To move the plunger rod into its initial position, the magnetic means and the magnetic counter-means co-operate magnetically in at least one position of the plunger rod. The magnetic means and the magnetic counter-means may co-operate with one another in several or in all positions which the plunger rod is able to assume, e.g., in a position which the plunger rod assumes when a product contained in the ampoule has been completely dispensed, so that the plunger rod can be moved back into its initial position. For example, the magnetic means may be disposed between the magnetic counter-means and the plunger, in which case the magnetic means and magnetic counter-means point to one another by the same poles in this arrangement so that the magnetic counter-means and hence the plunger rod is moved by the magnetic means in the direction of the initial position of the plunger rod. In another embodiment, the magnetic counter-means may be disposed between the magnetic means and plunger, in which case the magnetic means attracts the magnetic counter-means and hence the plunger rod in the direction of the initial position of the plunger rod in this arrangement. In some preferred embodiments, the magnetic means fixes the magnetic counter-means in its initial position, in which case the magnetic means and the magnetic counter-means are in contact or almost in contact, for example. In another embodiment of the invention, several magnetic means and/or magnetic counter-means are provided, which can co-operate with one another in a manner similar to that described above.

In a preferred embodiment, an engaging means is provided, which engages in the plunger rod and is able to prevent a movement of the plunger rod due to the magnetic forces. For example, the engaging means may be coupled with a dose-metering element, in which case a movement of the dose-metering element causes the plunger rod to be moved in the longitudinal direction or in the direction of the plunger, for example to set a dose or to administer a product contained in the ampoule. In some preferred embodiments, a rotation of the dose-metering element, for example to set a dose, causes a rotation of the engaging means, and/or a longitudinal movement of the dose-metering element, for example with a view to dispensing, causes a longitudinal movement of the engaging means. The plunger rod and the engaging means may have respective threads which are able to co-operate and form a threaded drive, for example, in order to convert the rotating movement of the engaging means into the longitudinal movement of the plunger rod. The engaging means may be a threaded nut, for example, e.g., a split threaded nut or part of a split threaded nut. For details of a split threaded nut and how the plunger rod is released, reference may be made to German published patent DE 102 29 138 A1 owned by the owner of the present application. In some embodiments, the engaging means is designed so that it is able to release the plunger rod to change the ampoule or re-set the plunger rod, so that the magnetic forces are able to move the plunger rod into its initial position. The engaging means may engage with and disengage from co-operating wedge-shaped elements, for example, by means of which the plunger rod is guided. The engaging means may have a wedge-shaped surface which is able to co-operate with a locking element. The locking element may also have a wedge-shaped surface. A wedge-shaped surface may cause an axial movement of the locking element to result in a radial movement of the engaging means. The locking element may advantageously be moved so that it radially overlaps with the engaging means to hold the engaging means engaged with the plunger rod.

In some preferred embodiments, the plunger rod is accommodated by the housing and/or the ampoule is accommodated by an ampoule holder which can be connected to the housing. For example, the housing and the ampoule holder have at least one means which is coupled with the engaging means so that when the housing and ampoule holder are connected, the engaging means sits in engagement with the plunger rod and when the housing and ampoule holder are released from one another, the engaging means is released from the plunger and the plunger rod is released. Consequently, when the housing parts are released from one another, the plunger rod can be moved back into its initial position.

In some embodiments, proximally of the ampoule holder, a longitudinally displaceable sleeve may be disposed on the housing which drives the locking means with it when the ampoule holder is attached to the housing, as a result of which the locking means is moved into engagement with the plunger rod. The sleeve and/or the locking means oppose a spring force when the ampoule holder is attached to the housing.

In another embodiment of the present invention, the magnetic means and the magnetic counter-means may be spaced at such a distance apart from one another that the plunger rod can not be moved back into the initial position in every position of the plunger rod. In this case, the plunger rod is moved into the initial position with the aid of gravity when the engaging means is released or moves back into a position in which the magnetic force is able to move the plunger rod. In one preferred embodiment, the magnetic counter-means is disposed at the proximal (rear) end of the plunger rod and the magnetic means is disposed at the proximal (rear) end of the injection pen or injection device. The magnetic means and magnetic counter-means may engage in the initial position so that they are not able to come apart from one another due to their natural weight, even if the force of their weight is opposing the magnetic attraction force.

In some embodiments, a cap may be attached to the distal (front) end of the housing, for example, which is able to cover the ampoule holder and a needle which can be attached to the ampoule or ampoule holder. The cap may have a clip, by means of which the injection device can be secured in a shirt pocket, for example.

The present invention encompasses a method of re-setting a plunger rod of an injection device wherein the plunger rod is firstly released to effect a movement along its longitudinal axis. A magnetic force is also exerted by one of a magnetic means and magnetic counter-means on the other of the two to enable the plunger rod to move back into its initial position. The method additionally involves the return movement of the plunger rod into the initial position and the fixing of the plunger rod in the initial position. In one aspect, the method is implemented due to the fact that the plunger rod engages with an ampoule or the ampoule is released or prevented from being released by the plunger. Advantageous method steps are also obtained from the use of the injection device described above.

### BRIEF DESCRIPTION OF THE DRAWINGS

FIG. 1 shows an embodiment of an injection device in accordance with the present invention prior to administering a product contained in an ampoule,

FIG. 2 shows the injection device illustrated in FIG. 1 after administering a product contained in the ampoule, and

FIG. 3 shows a detailed view of a portion of the injection device illustrated in FIG. 1.

### DETAILED DESCRIPTION

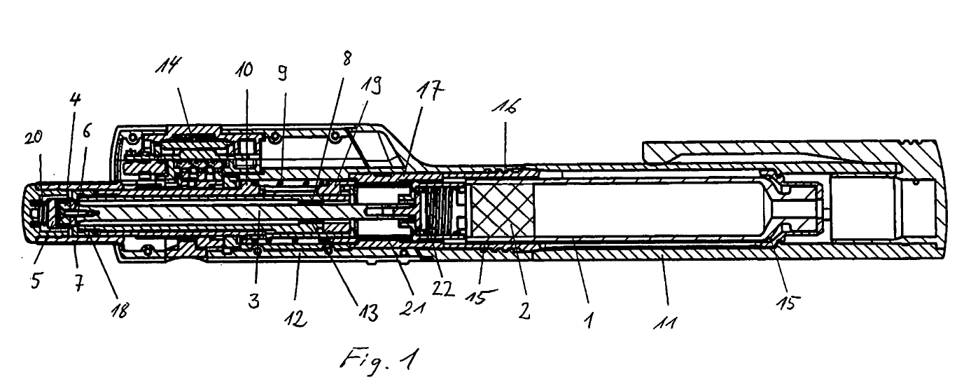

The injection pen illustrated in FIGS. 1, 2 and 3 represents one preferred embodiment of the present invention. The injection pen comprises a housing 12 and a cap 11 attached to it, which can be removed from the housing 12 and simultaneously forms a cover for an ampoule holder 15 or an ampoule 1 or a needle (not illustrated) which can be attached to it. The proximal housing part 11 may be affixed to the housing 12 by means of a thread or a catch means, for example. A plunger rod 3 is mounted on the housing 12 so that it is able to effect a longitudinal movement. The plunger rod 3 has a cylindrical portion, on which a thread extends. Attached to the distal end face of the plunger rod 3, for example by a thread, is a dish-shaped plate element. The plate element may co-operate with a plunger 2 which is disposed so as to be longitudinally displaceable in an ampoule 1 and seals off what is a liquid product contained in the ampoule in the proximal direction. Disposed at the proximal end of the plunger rod 3 is an annular magnetic counter-means 4, which is attached to the proximal end of the plunger rod 3 by means of a countersunk screw 6. The magnetic counter-means 4 may be a magnet for example, such as a permanent magnet or a ferro-magnet. The plunger rod 3 is secured to prevent it from rotating relative to the housing 12.

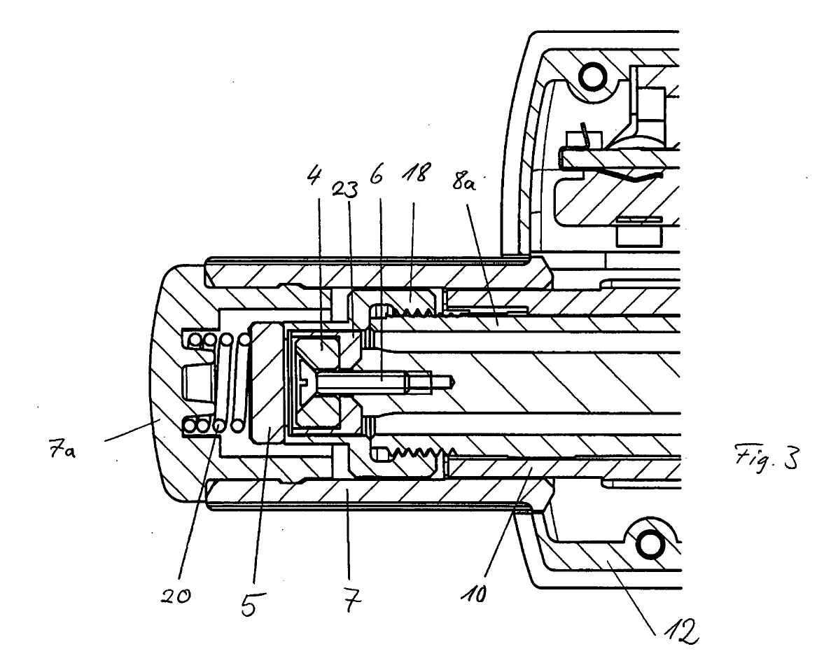

An engaging element 8 which is able to engage in the plunger rod 3 is also accommodated by the housing 12. As illustrated in FIG. 1, the engaging element 8 engaging with the plunger rod 3 and the plunger rod 3 form a threaded drive 13, which converts a rotating movement of the engaging element 8 into a longitudinal movement of the plunger rod 3. The engaging element 8 is disposed at the distal end of a sleeve-shaped element 8a (FIG. 3), which surrounds the plunger rod 3 in an initial position. Screwed onto the sleeve 8a at the proximal end of the sleeve is a threaded nut cap 18. The engaging element 8 together with the sleeve 8a and the threaded nut cap 18 is able to rotate and move axially relative to the housing 12. By means of a spring 9 acting on a transmission sleeve 10, the engaging element 8, sleeve 8a, threaded nut cap 18 and transmission sleeve are pushed in the proximal direction. As illustrated in particular in FIG. 3, the threaded nut cap 18 constitutes a proximal axial stop for the magnetic counter-means 4 and for the guide sleeve 23. To this end, the threaded nut cap 18 has a collar pointing radially inwards at its proximal end. The guide sleeve 23 surrounds the magnetic counter-means 4 at its periphery. The guide sleeve 23 also projects proximally (i.e., rearwardly) beyond the end face of the magnetic counter-means 4 so that the guide sleeve 23 is able to move into abutment with the threaded nut cap 18 to protect the magnetic counter-means 4 from excessive vibrations, which could cause damage to the magnetic counter-means 4. Alternatively or in addition, the magnetic counter-means 4 could be provided with a protective coating on its end face, e.g., a coating with a damping effect and, in some preferred embodiments, an elastomeric coating, to protect the magnetic counter-means 4 from excessive vibrations.

A dose-metering element 7 is accommodated at the proximal end of the housing 12 so that it can rotate and move longitudinally. The dose-metering element 7 is coupled with the transmission sleeve 10 so as to be fixed therewith in rotation, and the transmission sleeve 10 is joined to the sleeve 8a constituting the engaging means 8 so as to be fixed in rotation with it. When the dose-metering element 7 is moved in rotation, the engaging means 8 is also displaced in a rotating movement, as a result of which the plunger rod 3 is moved in the distal direction or in the direction of the plunger 2 depending on the desired dose.

The dose-metering element 7 accommodates a magnetic means 5, which co-operates in an attraction relationship with the magnetic counter-means 4 of the plunger rod 3. The magnetic means 5 may be a magnet for example, a permanent magnet, or may be made from a ferromagnetic material. A spring 20 is also accommodated in the dose-metering element 7, which is supported on the magnetic means 5 at one end and on the dose-metering means 7 or a closure cap 7a (FIG. 3) attached to the dose-metering element 7 at the other end. As a result, the closure cap 7a is held at a distance from the threaded nut cap 18 on the one hand and the magnetic means 5 is held in abutment with the threaded nut cap 21 on the other hand. The advantage of this application is that if the plunger rod 3 is returned quickly causing an impact, the spring 20 is able to absorb or damp the impact. This damping action in the end position is gentle on the magnet.

The transmission sleeve 10 has means on its outer periphery by which the relative angular position of the dose-metering element 7, transmission sleeve 10 or engaging means 8 can be fixed relative to the housing 12 by a dose display module 14. Due to the thread pitch of the threaded drive 13, the dose display module 14 is able to determine, on the basis of the relative angular position, the distance by which the plunger rod 3 was pushed in the direction of the plunger 2 to set a dose. Due to the internal diameter of the ampoule 1 and the dose-metering distance covered by the plunger rod 3, the dose display module 14 is able to display to the user the set quantity of product to be dispensed, e.g., on an LED element.

The ampoule 1 is accommodated by an ampoule holder 15. The ampoule holder 15 has a window, by means of which a check can be made so see how full the ampoule is. The ampoule holder 15 is releasably connected to the housing 12 by means of a thread connection 16. A clamping element 22 is also provided, which is pushed by a spring 21 against the ampoule 1, which spring 21 is supported by its one end on an element which is essentially axially stationary relative to the housing 12 and by its other end on the clamping element 22. Due to the axial force applied by the spring 9 via the clamping element 22 to the ampoule 1, the ampoule 1 is pushed into a distal (frontwardly) abutment with the ampoule holder 15.

The housing 12 also provides a mount for a sliding sleeve 17 and a release element 19, which can be moved longitudinally. The release element 19 is biased or urged in the distal (forward) direction by a force applied by the spring 9, which is supported on it and on the transmission sleeve 10. When an ampoule holder 15 is attached to the housing 12, the sliding sleeve 17 is pushed in a proximal direction and thus pushes on the release element 19 against the spring force of the spring 9. When the ampoule holder 15 is in the screwed-in state, the release element 19 radially overlaps with the engaging means 8 so that the engaging element 18 engages in the plunger rod 3 and thus forms a threaded drive 13.

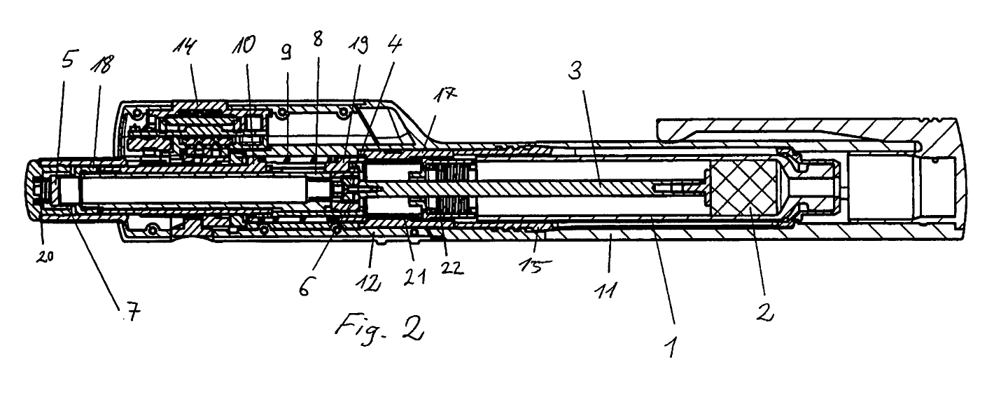

When the ampoule holder 15 is released from the housing 12, the release element 19 and the sliding sleeve 17 are pushed in the distal direction by the spring force of the spring 9. As a result, the release element 8, which is such that it has a biasing force acting radially outwards, moves radially outwards so that the engagement of the engaging element 8 with the plunger rod 3 is released. The plunger rod 3 is now released, so that a movement of the plunger rod 3 is possible in the longitudinal direction. In a corresponding position, the plunger rod 3 can be returned by force of gravity into its initial position illustrated in FIG. 1 and secured due to the co-operation of the magnetic means 5 and magnetic counter-means 4. The force of attraction from the magnetic means 5 and magnetic counter-means is sufficiently strong that the plunger rod 3 can no longer be moved out of its initial position by its own weight. In one embodiment, it can no longer be moved out of its initial position as long as the engaging means 8 is engaged. The plunger rod 3 can also be pulled back by the force of attraction of the magnetic means 5 and magnetic counter-means 4 from the most distal position of the plunger rod 3 illustrated in FIG. 2 if the magnetic fields of the magnetic means and magnetic counter-means are strong enough. In this case, gravitational force would no longer be needed to assist the return movement of the plunger rod 3 into its initial position or would merely support it.

Prior to dispensing a product for the first time, the plunger rod 3 is in its initial position (FIG. 1). By turning the dose-metering element 7, the plunger rod 3 is moved by the desired dose in the direction of the plunger 2 of the ampoule 1. The user of the device can remove the cap 11 from the pen before or after setting the desired product dose. Once the product dose has been set, the user of the injection pen injects the needle, not illustrated, into a desired body part. The user then pushes the dose-metering element 7 in the distal (forward or front) direction, as a result of which the dose-metering element 7 moves the threaded nut 18, the sleeve 8a forming the engaging means 8, the engaging means 8 and the transmission sleeve 10 in the dispensing direction. Since the plunger rod 3 and the engaging means 8 are engaged in the position illustrated in FIG. 1, when the engaging element 8 is moved longitudinally, the plunger rod 3 moves with it in the distal direction and the plunger 2 moves accordingly in the dispensing direction by the pre-set dose-metering distance. A dose of the product contained in the ampoule 1 can be metered and administered several times until the ampoule 1 is empty and the plunger 2 has reached the distal end face of the ampoule 1. In order to change the ampoule 1, the ampoule holder 15 is removed form the housing 12 and the spent ampoule 1 replaced with a new ampoule 1. As the ampoule holder 15 is removed from the housing 12, the engaging means 8 is radially released by the release element 19 so that the engaging means releases the plunger rod 3 and the plunger rod 3 is moved back into its initial position due to its force of gravity and/or magnetic force, where it can be secured. When the ampoule holder 15 is attached to the proximal housing part 12, the release element 19 is moved back so that it radially overlaps with the engaging means 8, so that the injection pen is ready for administering.

Embodiments of the present invention, including preferred embodiments, have been presented for the purpose of illustration and description. They are not intended to be exhaustive or to limit the invention to the precise forms and steps disclosed. Obvious modifications or variations are possible in light of the above teachings. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. An injection device which can accommodate an ampoule, the injection device comprising:
a) a plunger rod which can be moved out of an initial position in a dispensing direction to act on a plunger of the ampoule,
b) magnetic means, whereby the plunger rod can be moved along its longitudinal axis relative to the magnetic means,
c) magnetic counter-means disposed on the plunger rod, wherein
d) the magnetic means and the magnet counter-mans magnetically co-operate in at least one position of the plunger rod.

2. The injection device as claimed in claim 1, wherein the magnetic means and the magnetic counter-means can attract or repel.

3. The injection device as claimed in claim 1, wherein the magnetic means and the magnetic counter-means co-operate with one another in every position of the plunger rod, including in a position which the plunger rod assumes when a product contained in the ampoule has been completely dispensed, so that the plunger rod can be moved back into its initial position.

4. The injection device as claimed in claim 1, wherein at least one of the magnetic means and magnetic counter-means is a magnet, in particular a permanent magnet.

5. The injection device as claimed in claim 1, wherein if one of the magnetic means and magnetic counter-means is a permanent magnet, the other is a ferro-magnet.

6. The injection device as claimed in claim 1, further comprising a housing to which the ampoule is connected so as to be axially fixed in one direction, the magnetic means being axially stationary with respect to the housing.

7. The injection device as claimed in claim 1, wherein the magnetic counter-means is operably associated with the plunger rod.

8. The injection device as claimed in claim 7, wherein the magnetic counter-means is one of screwed to, adhered to or enclosed in the plunger rod.

9. The injection device as claimed in claim 1, wherein the plunger rod comprises the magnetic counter-means.

10. The injection device as claimed in claim 1, further comprising a dose-metering element moveable in rotation to set a dose or to administer a product contained in the ampoule, wherein the plunger rod can be moved in the longitudinal direction when the dose-metering element is moved to set a dose or to administer a product contained in the ampoule.

11. The injection device as claimed in claim 1, further comprising a rotatably moveable engaging means for engaging the plunger rod and converting a rotating movement of the engaging means into a longitudinal movement of the plunger rod.

12. The injection device as claimed in claim 11, wherein the engaging means is a split threaded nut or part of a split threaded nut.

13. The injection device as claimed in claim 11, wherein, to move the plunger rod back, the engaging means releases the plunger rod so that the plunger rod can be moved back into its initial position.

14. The injection device as claimed in claim 12, wherein, to move the plunger rod back, the engaging means releases the plunger rod so that the plunger rod can be moved back into its initial position.

15. The injection device as claimed in claim 1, further comprising a housing and an ampoule holder, wherein the plunger rod is accommodated by the housing and the ampoule is accommodated by the ampoule holder, the housing and ampoule holder can be connected to one another, and when the housing and ampoule holder are released from one another, the plunger rod can be moved back into its initial position.

16. The injection device as claimed in claim 1, wherein the plunger rod is moveable into an initial position by its own weight.

17. The injection device as claimed in claim 1, wherein the magnetic counter-means is disposed at an end of the plunger rod.

18. The injection device as claimed in claim 1, wherein the magnetic means and the magnetic counter-means are in an attracting engagement in the initial position, which position is the rearward-most position of the plunger rod, sufficiently that they can not come apart due to the weight of the plunger rod.

19. An injection device comprising a plunger rod moveable from an initial position in a dispensing direction and having a longitudinal axis, a magnetic member whereby the plunger rod can be moved along the longitudinal axis relative to the magnetic member, a magnetic counter-member associated with the plunger rod, wherein the magnetic member and the magnet counter-member magnetically co-operate in at least one position of the plunger rod.

20. The injection device as claimed in claim 19, wherein the magnetic member and the magnetic counter-member co-operate with one another in every position of the plunger rod, including in a position which the plunger rod assumes when a product contained in the ampoule has been completely dispensed, so that the plunger rod can be moved back into its initial position.
